(19) United States
(12) Patent Application Publication
(10) Pub. No.: US 2020/0168332 A1
(43) Pub. Date: May 28, 2020

(54) DISPLAY SYSTEM FOR MEDICAL INFORMATION AND METHOD FOR GENERATING DISPLAY CONTENT

(71) Applicant: TAIPEI MEDICAL UNIVERSITY, Taipei City (TW)

(72) Inventors: Ray-Jade Chen, New Taipei City (TW); Yu-Sheng Lo, New Taipei City (TW); Lan-Ying Kang, Kaohsiung City (TW); Kuang-Hung Hsu, Hsinchu County (TW); Chao-Chi Chen, Taipei City (TW); Yu-Tao Chang, Yunlin County (TW)

(21) Appl. No.: 16/699,012

(22) Filed: Nov. 28, 2019

(30) Foreign Application Priority Data
Nov. 28, 2018 (TW) 107142490

(51) Int. Cl. G16H 40/67; H04N 7/18; H04N 5/445; G06T 7/00; G06T 7/246; H04N 5/232; G16H 10/60; G16H 30/20

(52) U.S. Cl. CPC G16H 40/67; H04N 7/181; H04N 5/44591; G06T 7/0012; G06T 7/246; G06T 2207/30232; G16H 10/60; G16H 30/20; G06T 2207/30088; G06T 2207/30201; H04N 5/23203

(57) ABSTRACT

Provided herein is a display system for medical information and a method for generating display content. The system is applicable to a medical institution for monitoring statuses of patient beds. The system includes a patient bed system and a central serving system. The central serving system receives data generated by medical instruments around the patient beds simultaneously. In the central serving system, the data is analyzed and compared with thresholds for classifying the statuses of the patients. After visualizing the data, an information display panel is incorporated to display the information with respect to each of the patient beds. The status at each patient bed can be expressed by various graphic charts and shown on the panel. The different degrees of the statuses of patients are shown with different display effects that can be provided for the medical staff to determine and deal with emergency situations immediately.

### CROSS-REFERENCE TO RELATED PATENT APPLICATION

This application claims the benefit of priority to Taiwan Patent Application No. 107142490, filed on Nov. 28, 2018. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The disclosure is related to a system for displaying medical information, and in particular to a display system for remotely monitoring medical information of patient beds and allowing the medical staff can react to the emergent event being monitored instantly, and a method for generating the display content thereof.

### BACKGROUND OF THE DISCLOSURE

Various medical instruments are disposed around a patient bed of a hospital for examining various conditions of patient. For example, in an intensive care unit (ICU) of the hospital, physiological monitors are provided for monitoring various vital signs of the patient. The physiological monitors are provided to monitor the patient's physiological signals such as electrocardiography (ECG), heartbeat, blood pressure, respiration, and/or blood oxygenation. Furthermore, a respirator can be used to monitor the patient's respiration status and assist the patient's breathing in order to improve blood oxygenation. Still further, a blood oxygen monitoring machine can be used to constantly monitor the patient's blood oxygenation or others.

The various physiological data under monitoring may be corresponded to specific medical information and vital signs.

The medical instruments are generally connected with a central computer at a nursing unit. Rules for alerting can be set in the instruments for the medical staff to obtain information concerning the patient through the monitors.

### SUMMARY OF THE DISCLOSURE

The disclosure is related to a display system for medical information and method for generating display content. The display system provides a display panel for notifying medical staff of statuses at patient beds. The system also utilizes a software process to automatically assess the physiological data under monitoring and to generate warnings for providing immediate assistance to the patients.

According to one embodiment, the display system primarily includes a patient bed system that includes various medical instruments around one or more patient beds. Each of the patient beds or rooms is required to be disposed with one or more medical instruments. A central serving system with a host and a database is provided. The database is used to store data relating to the patient bed and the patient's medical records relating to examination, inspection, medication, and imaging. The host receives monitoring physiological data generated by the various medical instruments around the patient beds via a communication circuit. The physiological data undergoes data analysis performed by the host so as to generate instant monitoring data. The monitoring data is visualized as the medical information displayed on a display panel.

In the method, when the central serving system receives the physiological data from a patient bed, a data analysis is performed on the physiological data for generating the monitoring data. The central serving system uses each of the split screens to show the medical information of each individual patient bed. Further, the central serving system utilizes a software process to constantly assess the medical information with one or more thresholds so as to form different degrees of information. The system utilizes various display effects to show the different degrees of medical information on the display interface.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The present disclosure will become more fully understood from the following detailed description and accompanying drawings.

FIG. 1 is a schematic diagram depicting a display system for medical information according to one embodiment of the disclosure;

FIG. 2 is another schematic diagram depicting the display system in one further embodiment of the disclosure;

FIG. 3 shows a flow chart describing a method for generating medical information in a display system in one embodiment of the disclosure;

FIG. 4 shows one further flow chart describing the method for generating medical information in the display system in one further embodiment of the disclosure;

FIG. 5 shows another flow chart describing the method for generating medical information in the display system according one embodiment of the disclosure;

FIG. 6 shows an example of display content displayed on a display panel in one embodiment of the disclosure;

FIG. 7 shows another example of display content displayed on the display panel in one embodiment of the disclosure;

FIG. 8 shows one further example of display content displayed on the display panel in one embodiment of the disclosure;

FIG. 9 shows another example of the display content displayed on the display panel of one embodiment of the disclosure; and

FIG. 10 is a schematic diagram depicting an operating interface for controlling a camera of a patient bed in one embodiment of the disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first”, “second” or “third” can be used to describe various components, signals or the like, which are for distinguishing one component/signal from another one only, and are not intended to, nor should be construed to impose any substantive limitations on the components, signals or the like.

The disclosure is related to a display system for medical information and a method for generating display content in the system. The display system allows medical staff to access the monitoring data relating to patient beds of a patient bed system via a display panel. The system provides monitoring of physiological data of patients and can instantly alert the medical staff of any abnormal condition. The system allows the medical staff to obtain the real-time information at the patient beds via the display panel and provides assistance. The display system is implemented by a computer system, a database, and a network. The system integrates physiological data generated by medical instruments of a patient bed system, and shows the physiological data in a visualized aspect.

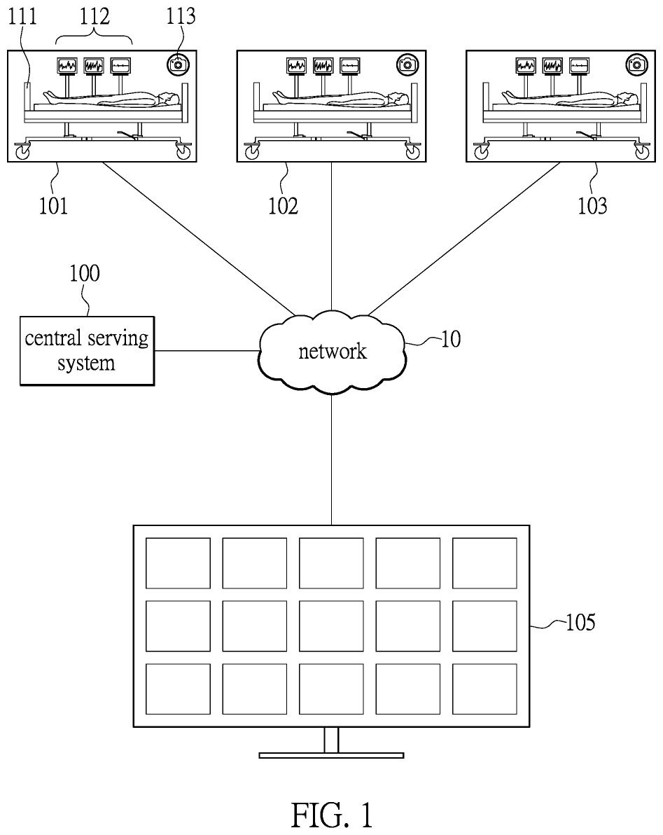

Reference is made to FIG. 1, which schematically shows a framework of the display system for medical information according to one embodiment of the disclosure.

The display system primarily includes a patient bed system in a medical institution. The patient bed system includes various medical instruments for one or more patient beds. In the diagram, a first patient bed 101 includes a patient bed 111, medical instruments 112, and a monitoring camera 113 at a side. Similarly, both a second patient bed 102 and a third patient bed 103 have the medical instruments or additional monitoring cameras.

The display system includes a central serving system 100 that includes a computer host and a database. The central serving system 100 can be disposed in a computer room of the medical institution. The central serving system 100 connects with the instruments of the patient bed system via a network 10, and receives the physiological data generated by the medical instruments via the network 10. After the data is processed, the medical information can be obtained and displayed on a display panel 105. The display panel 105 allows the medical staff to obtain patient conditions at different patient beds. The back-end automatic analysis and determination can instantly interpret the statuses of the patients.

The display panel 105 can be used to show real-time statuses of the patients through visualized graphic information. A software process running in a computer host of the central serving system 100 automatically compares the data generated by the medical instruments with thresholds. The comparison result is used to determine whether or not any abnormal condition occurs in the received physiological data. The abnormal condition can be shown on the display panel 105 and the related information is emphasized to the medical staff.

For example, when receiving heart rate data, which is a type of the physiological data that is monitored, the system provides a personalized alerting policy according to the basic data and examination data of the patient. It should be noted that the basic data of the patient is such as gender, age, height, weight, and/or medical history. The personalized alerting policy is applied to the physiological data for determining if any abnormal condition occurs. In general, every patient would have his own individual conditions that are used to form the personalized alerting policy for determining his personal abnormal conditions by incorporating various thresholds.

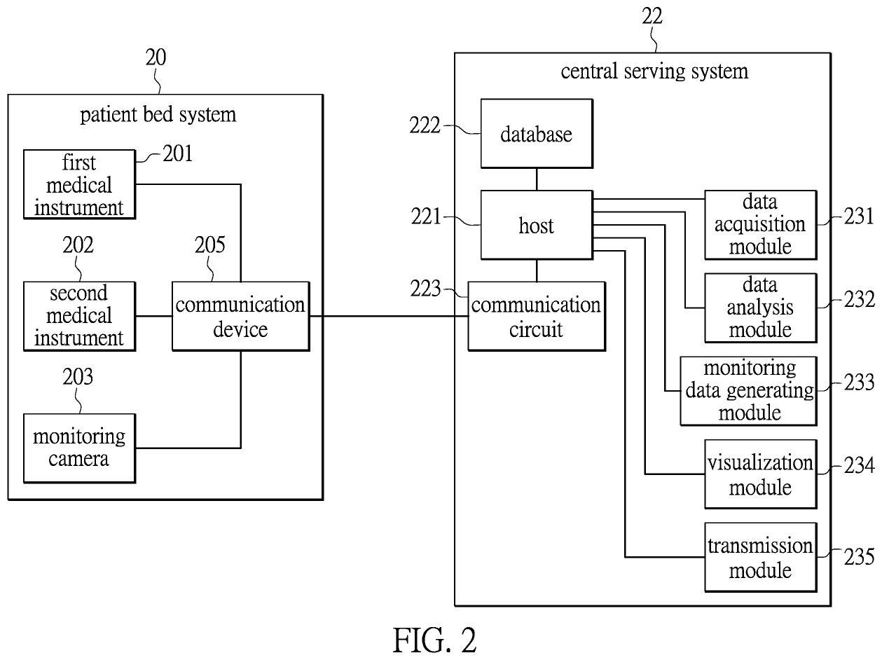

Reference is made to FIG. 2, which is another schematic diagram depicting framework of the display system in one embodiment of the disclosure.

The display system shown in the diagram includes a patient bed system 20 that includes various medical instruments such as a first medical instrument 201 and a second medical instrument 202. In one aspect of the system, a monitoring camera 203 is provided for capturing images within a patient room. In addition to monitoring activities in the patient room, the monitoring camera 203 can be used to capture the images of the patient's appearance, e.g. his face and skin. The system can determine if the patient is in a dangerous situation based on the status of the patient extracted from the images. It should be noted that the host of the central serving system performs an image-processing process to an image of each patient bed for obtaining image features that are used to determine facial or skin states of the patient, or for obtaining the patient's movement condition.

The patient bed system 20 has a communication device 205 that is electrically connected with the medical instruments within a patient room. The instruments are such as the first medical instrument 201, the second medical instrument 202 and the monitoring camera 203. The patient bed system 20 retrieves data generated by the medical instruments. The data can be transmitted to a central serving system 22 via a wired or wireless communication protocol afterwards, and a host 221 of the central serving system 22 receives the data by a communication circuit 223.

The central serving system 22 includes the host 221 and a database 222. The database 222 is used to store the patient data, the patient's historical data and examination data. The host 221 receives the physiological data generated by the one or more medical instruments (201, 202, and 203) in the patient room of the patient bed system 20 via the communication circuit 223. The physiological data and the images are analyzed in the host 221 for generating instant monitoring data. The instant monitoring data is visualized to generate the medical information displayed on a display panel.

According to one embodiment of the disclosure, the host 221 utilizes software to conduct data processing with the hardware such as a processor and a memory. A data acquisition module 231 is used to receive the physiological data from the patient bed system 20. The physiological data is associated with a patient room ID, a patient bed ID, a patient ID and a department classification, and is stored in the database 222. A data analysis module 232 is used to analyze the physiological data that is related to a patient room, a patient bed, and medical information of the patient.

The medical information is visualized as a graphical message by a visualization module 234 of the host 221. The graphical message is displayed on a display panel. The medical information of the patient can be shown with different graphical messages according to different degrees. A monitoring data generating module 233 is used to form the different graphical messages by comparing the medical information with one or more thresholds. A visualization module 234 is then used to show various display effects via a medical information display interface.

The host 221 further includes a transmission module 235 that can be used to transmit the patient's information to the people concerned, i.e. the patient's relatives, an attending doctor, and/or other doctors, by a simple message, a notification message, an electronic mail, or an instant message. The visualized medical information and the different degrees of messages processed by the monitoring data generating module 233 can be shown on a plurality of split screens via a medical information display interface on the display panel. Each of the split screens corresponds to the medical information of each of the patients.

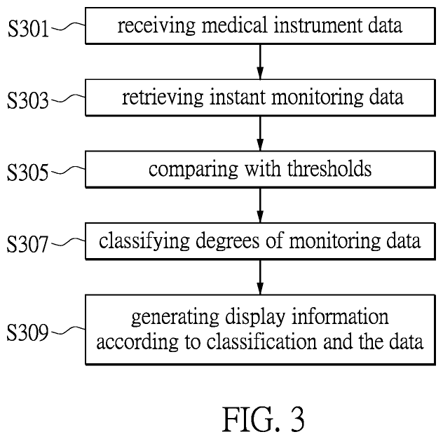

FIG. 3 shows a flow chart describing a method for displaying the medical information in one embodiment of the disclosure.

In step S301, a central serving system receives physiological data generated by one or more medical instruments in each of the patient beds via a network. In step S303, a data-processing means is used to retrieve the physiological data. In an exemplary example, the central serving system can receive physiological data from an ICU (Intensive Care Unit), and a software process running in the host determines critical medical information from the physiological data.

A software process operated in the central serving system defines one or more thresholds based on professional medical knowledge. The central serving system utilizes the thresholds to assess the physiological data by comparing with the physiological data (step S305). Each kind of physiological data has corresponding thresholds for classifying the data (step S307). For example, the physiological data is classified into multiple degrees of medical information indicating different levels of emergencies. The levels of emergencies can be expressed with different display effects through various colors, flashing lights and/or signal lights (step S309). After that, such as in step S309, the system shows the medical information with respect to every patient bed on the display panel based on the classification of the medical information and the physiological data.

The instant physiological data is such as a Heart Rate (HR). The related monitoring data can be shown by different colors indicating the emergency levels. For example, a color region with respect to HR is preset as having no background color, but then becomes red if the HR reaches a high level (e.g. HR>140), or yellow if the HR reaches a low level (e.g. HR<40). The high or low levels are defined beforehand in the system based on professional medical knowledge.

The physiological data is such as a Blood Pressure (BP). Similarly, a color region with respect to BP is preset as having no background color. A plurality of high and low thresholds are defined based on the judgment of a medical professional. The thresholds allow the system to generate various degrees of alerting signals. In an exemplary example with Systolic Blood Pressure (e.g. NBPs), the color region becomes red if NBP reaches a high level (e.g. NBPs>180). Alternatively, the color region becomes yellow if NBP reaches a low level (e.g. NBPs<90). In another example with Diastolic Blood Pressure (e.g. NBPd), a color region with respect to NBPd is preset as having no background color. The color region becomes red if NBPd reaches a high level (e.g. NBPd>90). Otherwise, if NBPd reaches a low level (e.g. NBPd<50), the color region is also red that indicates an emergency situation.

The instant physiological data indicating the patient's vital sign is such as Respiratory Rate (RR). A color region with respect to RR is preset to have no background color. Similarly, a plurality of high and low thresholds are defined based on the judgment of a medical professional. In this example, the color region becomes red if RR reaches a high level (e.g. RR>30/min), and is also red if RR reaches a low level (e.g. RR<8/min).

One further instant physiological data indicating the patient's vital sign is such as Oxygen Saturation (e.g. SpO₂). A default setting for a color region of SpO₂ is with no background color. According to the thresholds set by the medical professional, the color region can exemplarily be set as red when indicative of an emergency situation where SpO₂<90%.

Another instant physiological data indicating the patient's vital sign is such as Body Temperature (BT). A default setting for a color region with respect to BT is with no background color. Similarly, the medical professional defines the thresholds for BT. For example, the color region is set to be red if BT reaches a high level (e.g. BT>39 degree) or if BT reaches a low level (e.g. BT<34 degree).

For the patient's examination values, the system also defines thresholds for every examination item for assessing if the examination values reach the high or low thresholds.

Still further, a Sequential Organ Failure Assessment Score (SOFA Score) is used to automatically calculate a patient's score according to his respiration, blood, medicine and a coma index. The SOFA score gives scores within a range of 0 to 24, and with the aid of medical professionalism, a monitoring index is established to appropriately indicate the severity of a specific patient. The system can respectively utilize a red color, a yellow color or a green color when the patient is at a high risk (e.g. SOFA score≥10), a medium risk (e.g. SOFA score>6 and SOFA score<10), or a low risk (e.g. SOFA risk≤6) to distinguish between degrees of organ failure in each patient.

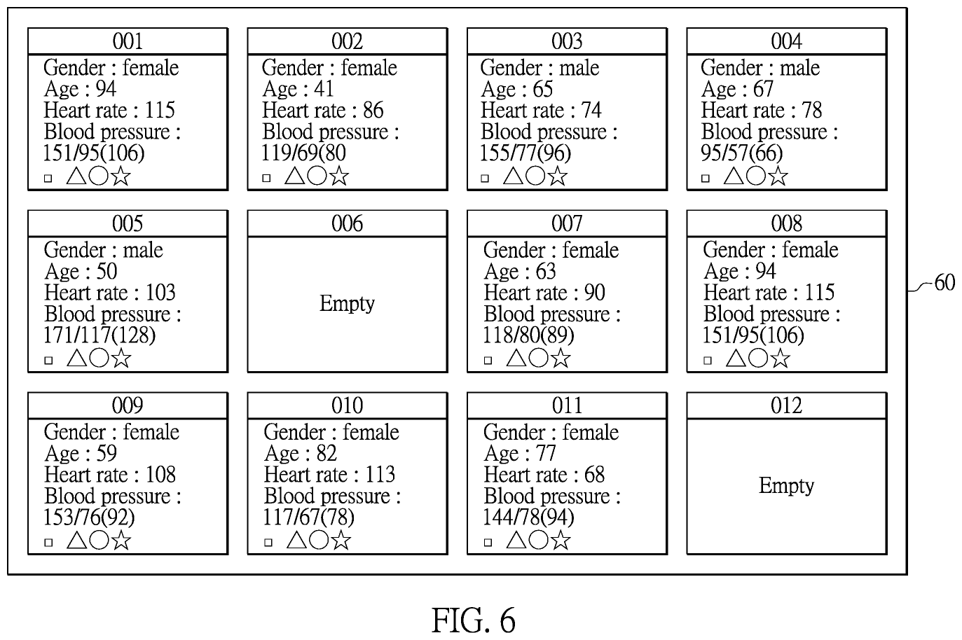

Reference is made to FIG. 6, which shows a schematic diagram describing a display system for medical information according to one embodiment of the disclosure. The diagram exemplarily shows a patient-centric dashboard.

A medical information display interface 60 is provided for showing information with respect to a plurality of patients in form of an array. The diagram shows each patient bed with one patient. The medical information display interface 60 shows medical information in connection with twelve patients by tag numbers 001 to 012. The patient bed numbers 006 and 012 are marked as empty beds.

In the present example, every split screen represents one patient and shows the patient's basic data, e.g. gender and/or age, which is retrieved from a database of the central serving system. The system allows the medical staff to choose the items to be displayed. Further, each split screen shows the medical information such as instant heart rate and blood pressure of one of the patients.

On the patient-centric dashboard, display effects such as colors, flashing lights and/or signal lights can be used to show the different levels of the statuses of the patients. The display effects allow the medical staff to acknowledge the situations of all of the patient beds.

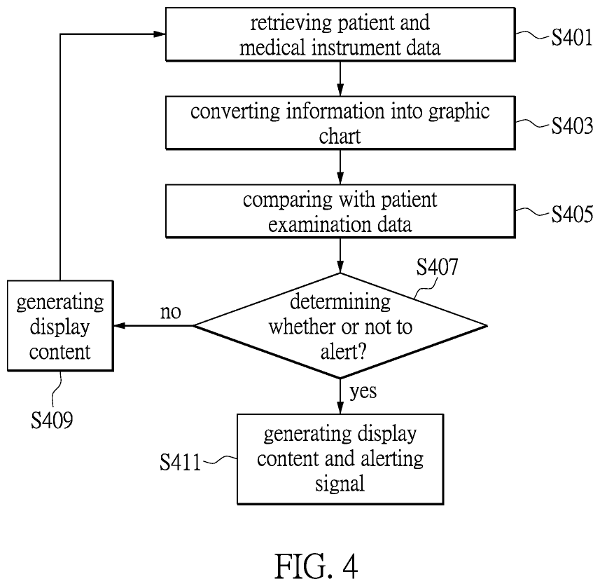

FIG. 4 shows a flow chart describing the method for generating display content in the display system according to one embodiment of the disclosure.

In the present example, in an initial step S401, a central serving system is provided to instantly obtain physiological data generated by medical instruments of every patient bed. In step S403, the physiological data is converted to a graphic chart. In addition to the graphic chart being displayed on a display panel, the physiological data can be compared with the thresholds set by doctors or the system according to each individual patient and with reference to the examination data in the database (step S405). In step S407, the system accordingly determines whether or not to generate an alerting signal.

If the physiological data does not reach an alerting threshold, in step S409, the data can be directly displayed on the display panel. Otherwise, if the physiological data reaches the threshold, such as in step S411, the system can utilize the display effects to show the alerting signal besides using the display panel to show the data. According to one of the embodiments, the system can inform relatives or the doctors of the patients in various ways if the alerting signal is acknowledged by the central serving system. The alerting signal allows the medical staff to be made aware of any abnormal item accurately and quickly. Therefore, the medical staff can provide medical assistances for the patients instantly and correctly based on the alerting signal.

In the flow charts shown in FIG. 3 and FIG. 4, the central serving system obtains the physiological data generated by the medical instruments of the patient beds. The system determines the weights for the various physiological data by referring to the patients' basic data and examination data in the database. Therefore, the system is capable of rendering a personalized alerting policy for each patient. Table 1 shows an example of the thresholds for SOFA scores.

Table 1 shows the SOFA scores 0 to 4. The index is determined based on the indexes such as Respiration, Coagulation, Liver Bilirubin, Cardiovascular Hypotension, Glasgow Coma Scale, and Urine output. The indexes are used to distinguish the degrees of organ failure of the patients.

TABLE 1

| | SOFA score | | | | |
|---|---|---|---|---|---|
| | 0 | 1 | 2 | 3 | 4 |
| Respiration | >400 | <400 | <300 | <200 | <100 |
| | | 221~301 | 142~220 | 67~141 | <67 |
| Coagulation | >150 | <150 | <100 | <50 | <20 |
| Liver | <1.2 | 1.2~1.9 | 2.0~5.9 | 6.0~11.9 | >12.0 |
| Bilirubin | | | | | |
| Cardio- | N/A | MAP | Dopamine | Dopamine | Dopamine |
| vascular | | <70 | ≤5 or | >5 or | >15 or |
| Hypotension | | | others | Norepine- | Norepine- |
| | | | | phrine | phrine |
| | | | | ≤0.1 | >0.1 |
| Glasgow | 15 | 13~14 | 10~12 | 6-9 | <6 |
| Coma Scale | | | | | |
| Urine output | <1.2 | 1.2~1.9 | 2.0~3.4 | 3.5~4.9 | >5.0 or |
| | | | | or <5.00 | <200 |

The monitoring data and definitions are as follows:

Respiration: Respiration PaO₂FlO₂ or SaO₂/FlO₂ (mmHg);

Liver Bilirubin: Liver Bilirubin (mg/dl);

Renal Creatinine: Renal Creatinine (mg/dl);

Urine output: Urine output (ml/d); and

Mean arterial pressure: Mean arterial pressure (MAP).

The central serving system constitutes a personalized alerting policy according to the concept of Table 1. When the data shows any abnormality based on the personalized alerting policy, the display panel displays an alerting signal. According to one embodiment of the display system for medical information of the disclosure, various thresholds are defined based on the Table 1. The physiological data generated in each patient bed is obtained via the central serving system, and a personalized alerting policy is provided according to each patient's basic data and examination data in the database for each of the patient beds. An alerting signal is generated if an emergency event is determined and the alerting signal is shown with an alerting effect via a medical information display interface.

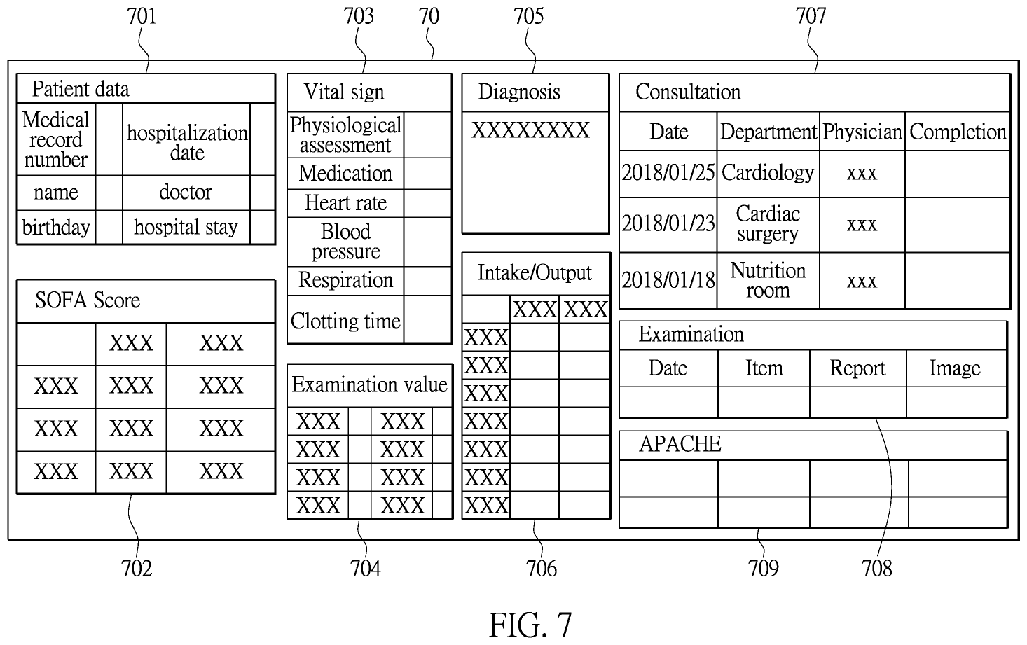

Next, referring to the schematic diagram shown in FIG. 7, a medical information display interface 70 shows medical information of one patient under an individual display mode out of multiple display modes. The medical information displayed on the medical information display interface 70 includes physiological data generated by the medical instruments and examination data of the patient. Several fields are shown in the medical information display interface 70. The patient data filled in the fields are such as a medical record number, a hospitalization date, a name, a doctor, a birthday and days of hospitalization. A SOFA score 702 is as the data shown in Table 1. Vital signs 703 can record physiological assessment, medication, heart rate, blood pressure, respiration, and clotting time. Examination value 704 records various examination data of the patient. Diagnosis 705 records historical diagnosis data of the patient. Intake/Output (IO) 706 is the measurement data made by medical staff, and abnormal values of IO recorded within the past three shifts will be shown. Consultation 707 records the records such as date, department, and doctor regarding the patients joining in consultation. Examination 708 records the past examination records of the patient and the records are such as dates, items, reports and images. Acute physiology and chronic health evaluation (APACHE) 709 marks a health status of a patient with several alarming states, e.g. a high threshold (red), a low threshold (red) and a normal threshold (green), in accordance with a physician's professional experiences.

Further, individual patients can also be marked with special notes via the medical information display interface 70. The special notes allow the medical staff to acknowledge every patient's characteristics quickly, and the medical staff can pay attention to these characteristics in a medical procedure. By default, no special note is provided to a patient. The central serving system can automatically retrieve the special notes for the patients and mark them with different icons. The special notes can be icons representing diagnosed conditions such as brain, heart, or liver conditions, or others such as requiring constraint, do-not-resuscitate (DNR), suicide prevention, pipeline safety, falls down easily, requiring isolation, and allergies.

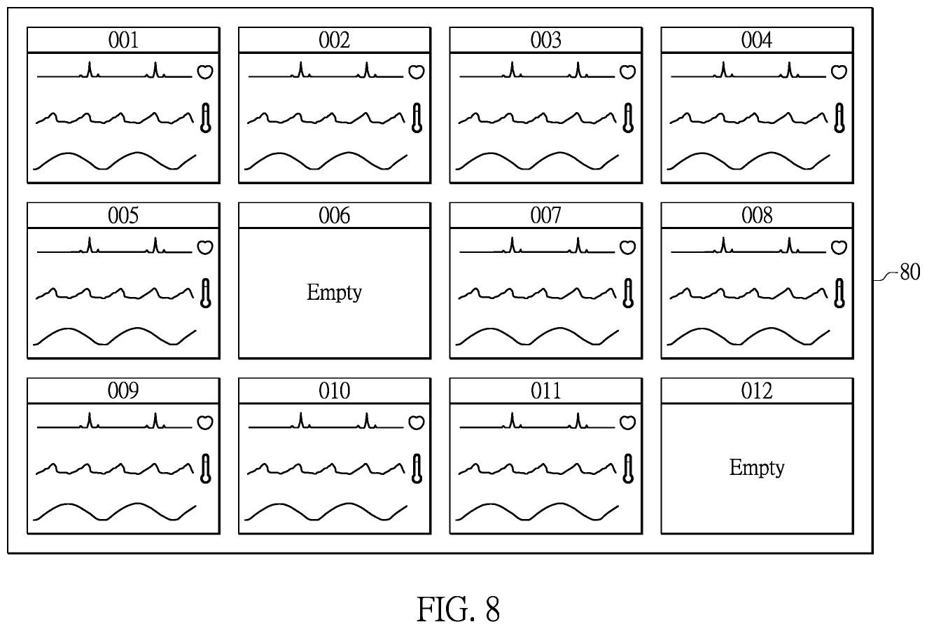

In one further embodiment, one of the display modes provided by the display system is a graphic chart display mode. FIG. 8 shows a schematic diagram depicting a display content of the display system.

A medical information display interface 80 showing a plurality of charts with curves with respect to patient bed or patient numbers 001 to 012. The central serving system retrieves physiological data generated by the medical instruments of the patient beds. The medical information extracted from the physiological data can be converted to a graphic chart. The graphic chart is used to show the information extracted from the physiological data. The present example shows the curves indicative of the real-time information such as heart rates, blood oxygenation and respiration of the patient beds. The medical information display interface 80 can also show the values such as body temperature and heartbeat.

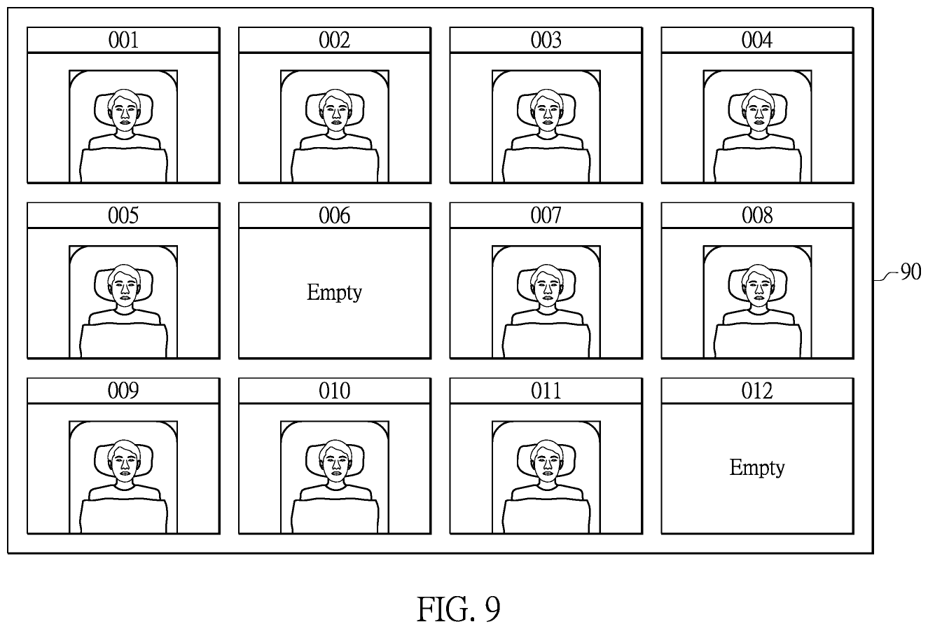

According to one embodiment of the disclosure, one of the display modes of the system is such as an image display mode. A plurality of split screens are shown on a display panel. The system utilizes the split screens to show the images with respect to the patient beds individually. Reference is made to FIG. 9, in which a monitoring camera is disposed in each of the patient rooms of the patient bed system. The monitoring camera is used to capture images of the patient room, including the patient bed. A communication device of the patient room is used to transmit the images to the central serving system.

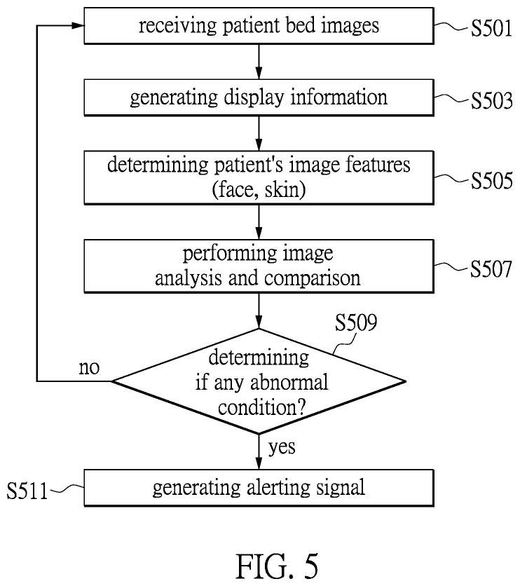

The relevant flow chart can be referred to in FIG. 5 according to one embodiment of the disclosure. In step S501, a host of the central serving system receives images of each of the patient beds. The images are converted to display content, such as in step S503. The images captured by the monitoring camera in each of the patient rooms are as the content displayed on the medical information display interface 90 according to the patient bed IDs or the patient IDs.

In the host of the central serving system, an image processing process is performed on the images of the patient beds. In step S505, features can be extracted from the images. The features are used to determine statuses of the faces or skins of the patients. The statuses of the faces or skins may relate to the colors, spots or shapes of faces or skins, or a movement state of each of the patients. In step S507, the statuses of the patients can be determined through the processes of image analyses and comparisons. In step S509, the system determines whether or not the patient is in an abnormal state. If the patient is in a normal state, the relevant image processing may continue, and the method proceeds to step S501 for continuously retrieving images and performing image processing. If the patient is diagnosed with abnormalities, such as in step S511, the system automatically generates an alerting signal.

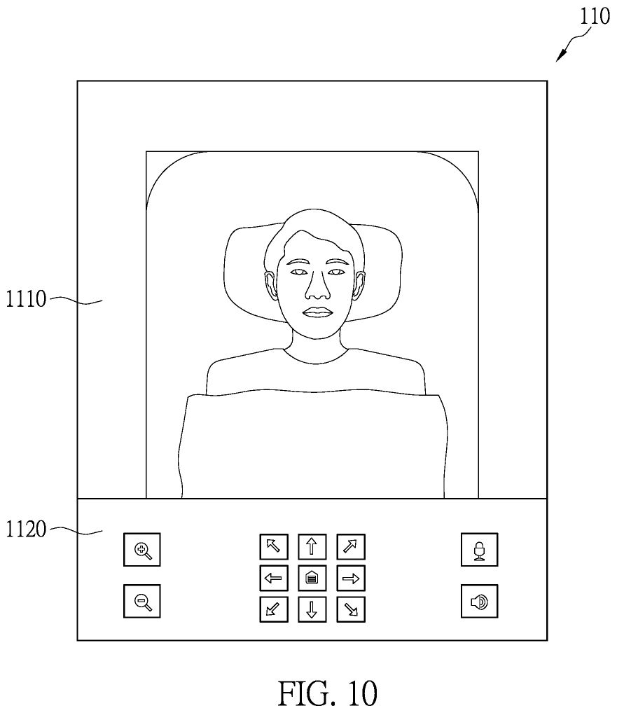

With the function of image monitoring proposed by the display system, as referring to FIG. 10, the display system provides a remote control function for allowing the medical staff to remotely control a monitoring camera in each of the patient rooms via an operating interface.

An image control interface 110 is exemplarily shown in the diagram. An upper portion of the screen shows a patient image 1110 taken by the monitoring camera. A lower portion of the screen shows a camera control panel 1120 that allows the medical staff or a user to remotely control zooming and rotation of the camera. Furthermore, the image control interface 110 also provides functions of remote voice communication and volume adjustment.

To sum up the above descriptions, according to the embodiments of the display system for medical information and method thereof, the system utilizes a display panel to show data generated by the medical instruments for allowing multiple patients to be monitored. The data is processed by data requisition and analysis for obtaining the critical medical information with respect to each of the patients. The system provides a database for storing patient data, historical data, and examination data. The system can instantly show the physiological data with each patient's basic data, historical data and examination data. A host of the system is used to receive the physiological data and then store, analyze and compare the physiological data with the thresholds for allowing the medical staff to integrate the medical information and make an instant determination and response.

The above-mentioned display panel can show the patient's real-time statuses by visualized graphs. For example, the system utilizes graphic charts under a display mode to represent various physiological data such as cardiograms, blood sugar levels, heart rates and body temperature. When compared with thresholds, an alerting signal will be shown on the display panel if any abnormal condition is determined. A monitoring camera and an image-processing technology can be used to acquire instant patient status by analyzing the images.

Thus, it is different from the general circumstance that requires medical staff to keep a vigilant eye on the medical instruments around the patient bed at any time, or requires the patient to call for the medical staff when encountering a sudden situation. The disclosed display system allows the medical staff to obtain comprehensive medical information by integrating the patients' examination data, medical histories, and their basic data. In particular, the medical staff can obtain important and real-time information about the patients from the graphical messages on the display panel.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A display system for medical information, comprising:
a patient bed system including one or more medical instruments at one or more patient beds; and
a central serving system including a host and a database, wherein the database stores patient data, historical data and examination data relating to each patient; the host receives physiological data generated by the one or more medical instruments of each of the patient beds in the patient bed system via a communication circuit; the physiological data form instant monitoring data after the host performs data analysis; the physiological data is visualized and becomes the medical information displayed on a display panel;
wherein each of the one or more patient beds has a communication device that is used to transmit the physiological data generated by the one or more medical instruments to the central serving system; the display panel displays a medical information display interface that uses split screens to respectively show medical information corresponding to each of the patients;
wherein the physiological data generated in each patient bed forms information of different degrees when compared with one or more thresholds, and the information is visualized on the medical information display interface with various display effects.

2. The system according to claim 1, wherein each of the patient beds of the patient bed system has a monitoring camera that is used to take images of the patient bed, and the images are transmitted to the central serving system by the communication device.

3. The system according to claim 1, wherein the physiological data generated in each of the patient beds is obtained via the central serving system, and a personalized alerting policy is provided according to basic data and examination data of each patient in the database at each of the patient beds; an alerting signal is generated if an emergency event is determined, and the alerting signal is shown by with alerting effect via the medical information display interface.

4. The system according to claim 3, wherein each of the patient beds of the patient bed system has a monitoring camera that is used to take images of the patient bed, and the images are transmitted to the central serving system by the communication device.

5. The system according to claim 1, wherein a plurality of display modes are applied to the medical information corresponding to each of the patients, and the display modes include a graphic chart display mode that converts the medical information to a graphic chart and utilizes the graphic chart to show the physiological data.

6. The system according to claim 5, wherein the display modes include an image display mode that utilizes the split screens to show images of the patient beds.

7. The system according to claim 6, wherein each of the patient beds of the patient bed system has a monitoring camera that is used to take images of the patient beds, and the images are transmitted to the central serving system by the communication device.

8. The system according to claim 7, wherein the display modes include an individual display mode that shows the medical information including the physiological data generated by the medical instrument and examination report of each of the patients via the medical information display interface.

9. The system according to claim 8, wherein each of the patient beds of the patient bed system has a monitoring camera that is used to take images of the patient bed, and the images are transmitted to the central serving system by the communication device.

10. The system according to claim 9, wherein the host of the central serving system performs an image-processing process to an image of each of the patient beds for obtaining image features that are used to determine a state of the face or skin from the image of the patient or obtain a movement state of the patient.

11. The system according to claim 10, wherein the host provides an image control interface on the display panel that is used to remotely control the monitoring camera.

12. A method for generating display content of medical information, wherein the method is applied to the display system according to claim 1, and the method comprising:
a host of a central serving system receiving physiological data generated by one or more medical instruments of a patient bed via a communication circuit;
generating instant monitoring data after the physiological data is analyzed by the host;
visualizing the monitoring data and generating medical information displayed on a display panel;
displaying a medical information display interface on the display panel, and each of a plurality of split screens of the display interface displaying medical information corresponding to each of patients; and
comparing the medical information with one or more threshold so as to form information of different degrees that are displayed on the medical information display interface with different display effects.

13. The method according to claim 12, wherein a plurality of display modes are applied to the medical information corresponding each of the patients, and the display modes include a graphic chart display mode that converts the medical information to a graphic chart and utilizes the graphic chart to show the physiological data.

14. The method according to claim 13, wherein the display modes include an image display mode that utilizes the split screens to show images of the patient beds, and an individual display mode that shows the medical information including the physiological data generated by the medical instruments and examination data of each of the patients via the medical information display interface.

15. The method according to claim 14, wherein each of the patient beds of the patient bed system has a monitoring camera that is used to take images of the patient bed, and the images are transmitted to the central serving system by a communication device.
